(19) United States
(12) Patent Application Publication
(10) Pub. No.: US 2023/0138134 A1
(43) Pub. Date: May 4, 2023

(54) HAIR STYLING SHEET DEVICES AND METHODS

(71) Applicant: Richard Morrill, Littleton, CO (US)

(72) Inventor: Richard Morrill, Littleton, CO (US)

(21) Appl. No.: 17/980,311

(22) Filed: Nov. 3, 2022

Related U.S. Application Data
(60) Provisional application No. 63/275,036, filed on Nov. 3, 2021.

(51) Int. Cl. A45D 34/04

(52) U.S. Cl. CPC A45D 34/04; A45D 2200/1027

(57) ABSTRACT

A hair styling sheet includes paper and a hair styling aid and/or haircare product. The paper may be parchment paper or wax paper. The hair styling aid or haircare product may be hair styling mousse, gel, hairspray, or wax product.

### CROSS-REFERENCES TO RELATED APPLICATIONS

This application claims the benefit of U.S. Provisional Patent Application No. 63/275,036 filed Nov. 3, 2021, the disclosure of which is incorporated herein by reference.

### BACKGROUND OF THE INVENTION

Embodiments of the present invention relate generally to the field of personal hair care, and in particular for devices for styling hair, as well as methods for their use and manufacture.

A wide variety of hair care products are available for use in styling one's hair or another person's hair. Yet still further improvements are desired. Embodiments of the present invention provide solutions to at least some of these outstanding needs.

### BRIEF SUMMARY OF THE INVENTION

Embodiments of the present invention encompass hair styling sheet devices and methods for their use and manufacture.

In one aspect, embodiments of the present invention encompass hair styling sheets methods for their use, and systems and methods for their manufacture. Exemplary hair styling sheets can include a paper component and a hair styling aid component. In some cases, a sheet can have a dimension of about 4 inches by about 6 inches. In some cases, a hair styling aid can include a hair styling mousse, a gel, a hairspray, or a hair wax product. In some cases, the hair styling aid includes a gel, and the gel includes a polymer. In some cases, the hair styling aid includes a gel, and the gel includes a cationic polymer. In some cases, the hair styling aid includes a gel, and the gel includes a copolymer.

In another aspect, embodiments of the present invention encompass methods of manufacturing a hair styling sheet which include placing a roll of paper on a dowel of a paper holder assembly, placing a fluid hair styling aid into a basin of a hair styling aid applicator assembly, contacting paper of the roll of paper with a roller of the hair styling aid applicator assembly, positioning paper of the roll of paper adjacent to a heating assembly, attaching a leading portion of the paper with a receiving roller, contacting paper of the roll of paper with the fluid hair styling aid which is in the basin of the hair styling aid applicator assembly, so as to provide a layer of the fluid hair styling aid onto the paper, thus producing a fluid aid coated paper, rotating the receiving roller so as to expose the fluid aid coated paper to the heating assembly and thereby produce a dehydrated aid coated paper, and so as to wrap the dehydrated aid coated paper around the receiving roller, removing the wrapped dehydrated aid coated paper from the receiving roller, and cutting a piece from the dehydrated aid coated paper, wherein the piece is the hair styling sheet. In some cases, the hair styling sheet is cut to a dimension of about 4 inches by about 6 inches. In some cases, the fluid hair styling aid includes a hair styling gel. In some cases, the fluid hair styling aid includes a hair styling gel, and the hair styling gel includes a polymer. In some cases, the fluid hair styling aid includes a hair styling gel, and the hair styling gel includes a cationic polymer. In some cases, the fluid hair styling aid includes a hair styling gel, and the hair styling gel includes a copolymer. In some cases, the fluid hair styling aid includes a hair styling gel, and the hair styling gel includes a viscosity modifier. In some cases, the viscosity modifier includes a carbomer. In some cases, the fluid hair styling aid includes a hair styling gel, and the hair styling gel includes a fragrance. In some cases, the fluid hair styling aid includes a hair styling gel, and the hair styling gel includes an ultraviolet protectant.

In some cases, manufacturing methods may include aligning the paper holder assembly and the hair styling aid applicator assembly relative to one another so that a longitudinal axis of the dowel of the paper holder assembly and a longitudinal axis of the roller of the hair styling aid applicator assembly are in parallel relationship with each other. In some cases, manufacturing methods may include aligning the paper holder assembly and a dowel of the receiving roller relative to one another so that a longitudinal axis of the dowel of the paper holder assembly and a longitudinal axis of the dowel of the receiving roller are in parallel relationship with each other. In some cases, manufacturing methods may include aligning the hair styling aid applicator assembly and the receiving roller relative to one another so that a longitudinal axis of the roller of the hair styling aid applicator assembly and a longitudinal axis of a dowel of the receiving roller are in parallel relationship with each other. In some cases, manufacturing methods may include aligning the paper holder assembly, the hair styling aid applicator assembly, and the receiving roller relative to one another so that a longitudinal axis of the dowel of the paper holder assembly, a longitudinal axis of the roller of the hair styling aid applicator assembly, and a longitudinal axis of a dowel of the receiving roller are in parallel relationship with each other.

### BRIEF DESCRIPTION OF THE DRAWINGS

FIG. 1 depicts aspects of a hair styling sheet manufacturing system, according to embodiments of the present invention.

FIG. 2 depicts aspects of a hair styling sheet manufacturing system, according to embodiments of the present invention.

FIG. 3 depicts aspects of a hair styling sheet manufacturing system, according to embodiments of the present invention.

FIG. 4 depicts aspects of a hair styling sheet manufacturing system, according to embodiments of the present invention.

FIG. 5 depicts aspects of a hair styling sheet manufacturing system, according to embodiments of the present invention.

FIGS. 6A and 6B depict aspects of a hair styling sheet manufacturing system, according to embodiments of the present invention.

FIG. 7 depicts aspects of a hair styling sheet manufacturing system, according to embodiments of the present invention.

FIG. 8 depicts aspects of a hair styling sheet manufacturing system, according to embodiments of the present invention.

FIG. 9 depicts aspects of hair styling sheet devices, according to embodiments of the present invention.

FIG. 10 depicts aspects of hair styling sheet devices, placed in a packet, according to embodiments of the present invention.

FIG. 11 depicts aspects of hair styling sheet devices and methods for their use, according to embodiments of the present invention.

### DETAILED DESCRIPTION OF THE INVENTION

Embodiments of the present invention encompass hair styling sheets which are configured to deliver haircare products and hair styling aids to a person's hair. In some cases, hair styling sheet embodiments can use paper sheets which may include parchment paper or wax paper to apply hair styling aids or other styling products. In this way, paper sheets can operate as the main delivery system. When the sheet devices are applied on or contacted with a person's hair, which may be either wet hair or dry hair, there is a transfer of the haircare product or hair styling aid from the paper to the person's hair. A paper sheet device can include a paper sheet in combination with a haircare product or hair styling aid. Exemplary haircare products and/or hair styling aids include hair styling mousse, gel, hairspray, wax products, and the like. In some cases, paper sheet devices can be manufactured by applying the haircare product and/or hair styling aid to paper, drying the combined product and/or aid, and then cutting the dried combined product and/or aid into sheets. For example, the sheets may be 4 inches by 6 inches in dimension. Sheets may also be provided in other sizes as well.

According to some embodiments, the product and/or aid can be applied by drawing rolls of paper through the product and/or aid. The resulting combination of paper along with the product and/or aid can then be dried, and cut into sellable pieces. The pieces can be placed into packaging prior to sale, shipment, or storage.

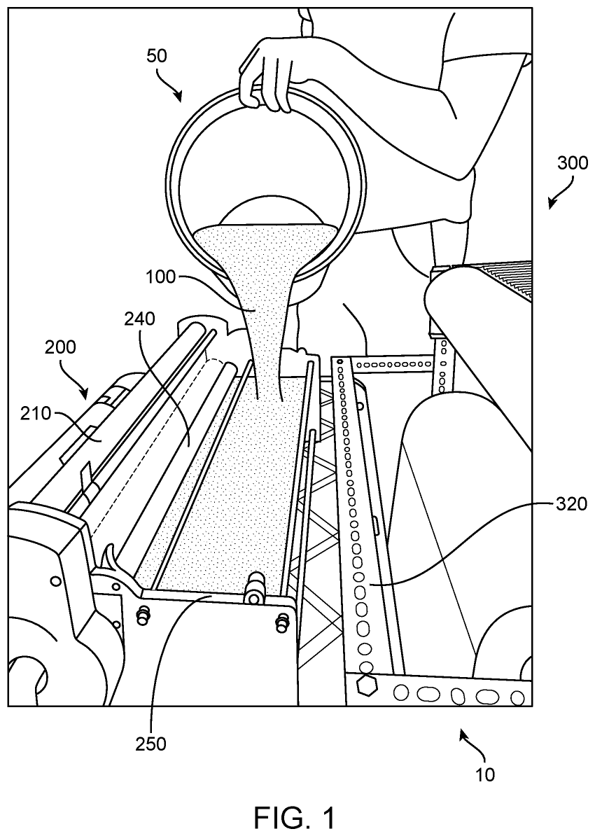

Turning now to the drawings, FIG. 1 illustrates aspects of method for manufacturing a hair styling sheet. The sheet can include paper and a hair styling aid. As shown here, a hair styling aid 100 can be placed into a hair styling aid applicator assembly 200. In this embodiment, the hair styling aid 100 is poured from a container 50 into a basin 250 of the hair styling aid applicator assembly 200. The consistency of the hair styling aid 100 can be adjusted, for example by adding water to the container 50 prior to pouring the aid into the basin 250. In some cases, the hair styling aid can include a hair gel such as a hard rock gel, or an extreme holding styling gel. The applicator assembly 200 includes a roller 210 about which the paper (not shown) is routed. The applicator assembly 200 is positioned adjacent to a paper holder assembly 300. As shown here, the paper holder assembly 300 includes a guide bar 320, which in operation can engage or contact the applicator assembly 200 so as to ensure that the roll of paper and components of the applicator assembly (e.g. basin 250, roller 210) remain aligned in parallel. This alignment can ensure proper application of the gel to the paper. In some embodiments, the applicator assembly 200 may include an adjustable thickness mechanism 240 which can operate to adjust the thickness of the gel which is applied to the paper. In some instances, the applicator assembly 200 and paper holder assembly 300 can be part of a manufacturing system 10.

In exemplary embodiments, the hair styling aid 100 includes one or more components of a hair gel. The hair styling aid 100 adheres to the paper as the paper travels through the applicator assembly. The hair styling aid may be in a fluid or flowable state. Some gels provide a greater hold on the hair and make the hair more stiff, while other gels provide less hold and make the hair not as stiff. In some cases, the aid 100 may include a colorant. In some cases, the aid may include a cationic polymer. In some cases, the aid may include a copolymer, such as a copolymer of vinylpyrrolidone, methacrylamide, and N-vinylimidazole. In some cases, the aid 100 may include water. In some cases, the aid may include a viscosity modifier, such as a carbomer. In some cases, the aid may include a fragrance. In some cases, the air may include an ultraviolet (UV) protectant. In some cases, the aid may include one or more of the following components: poly N-vinyl-2-pyrrolidone (PVP), polyvinyl acetate (PVA), vinyl pyrrolidone (VP), a PVP/PA copolymer, polyurethane, a vinyl monomer, glycerin, a stabilizer, a solvent, disodium EDTA, and a preservative. In some cases, the aid may include a hair styling mousse, a hairspray, or a wax product.

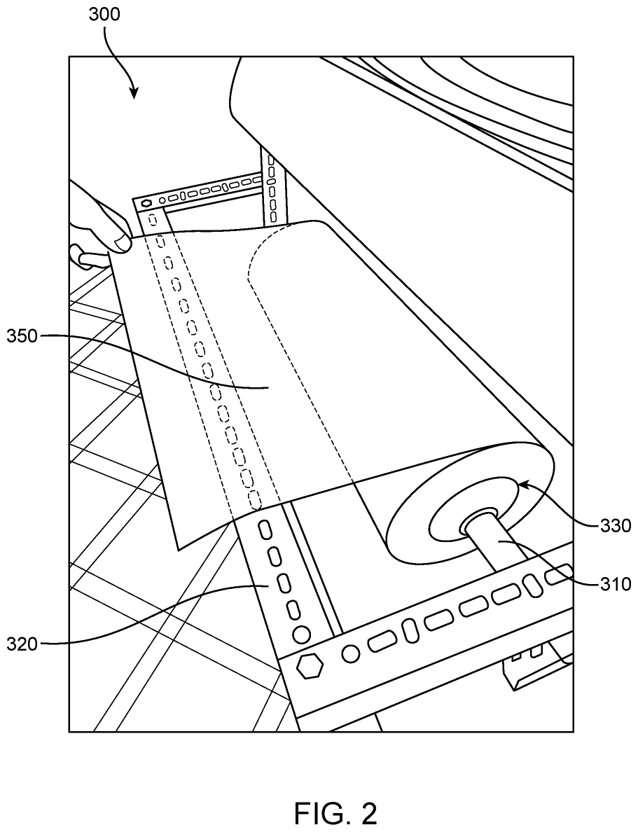

FIG. 2 depicts a roll of paper 350 engaged with a paper holder assembly 300. As shown here the paper holder assembly 300 includes a dowel 310 which holds the paper 350. The roll of paper which is to be coated with gel can be placed on the dowel 310. The paper holder assembly 300 can also include one or more lock mechanisms 330, for example engaged with the dowel 310 at one or both ends of the paper roll, so restrain the roll of paper from moving or translating along the long axis of the dowel. This can help to ensure alignment of the paper roll with other components of the system. In some instances, the paper 350 can be a wax paper. In some cases, the paper 350 can be a parchment paper. In some cases, the paper includes a thin layer of a non-stick material, such as a wax (e.g. paraffin wax) or silicone, which allows the hair styling aid to come off of the paper at the desired time. In some cases, the wax or silicone can be present on the outside of the paper, as a coating. In some cases, the wax or silicone can be present as an embedded material, within the pores of the paper. In some cases, the wax or silicone is embedded within the pores of the paper and also coats the outside of the paper.

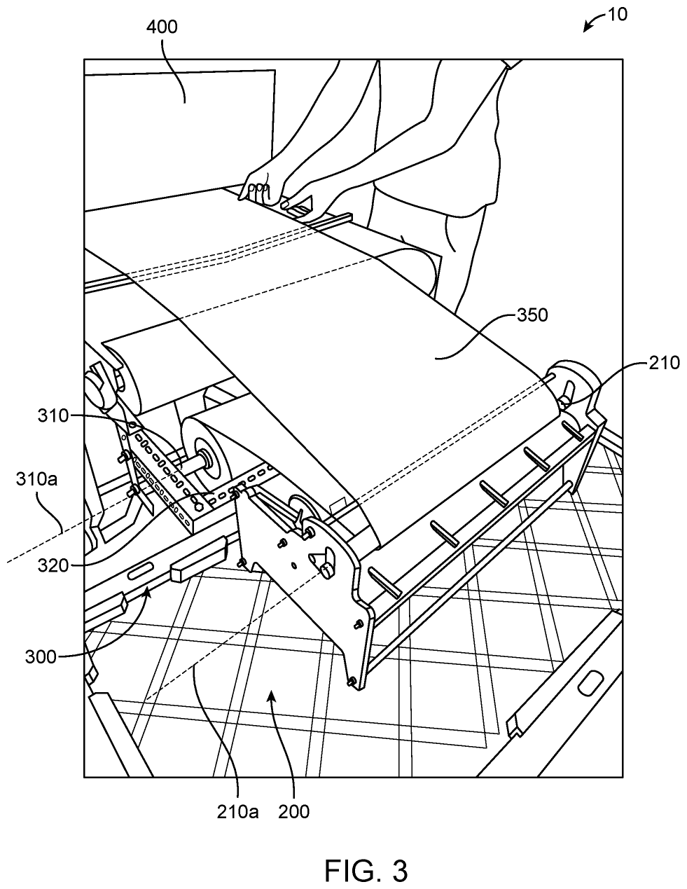

FIG. 3 shows the paper as it is routed through the applicator assembly 200, and toward a heating assembly 400. In some instances, the applicator assembly 200, paper holder assembly 300, and heating assembly 400 can be part of a manufacturing system 10. As illustrated here, the paper holder assembly 300 and the hair styling aid applicator assembly 200 can be aligned relative to one another so that a longitudinal axis 310a of the dowel 310 of the paper holder assembly 300 and a longitudinal axis 210a of the roller 210 of the hair styling aid applicator assembly 200 are in parallel relationship with each other.

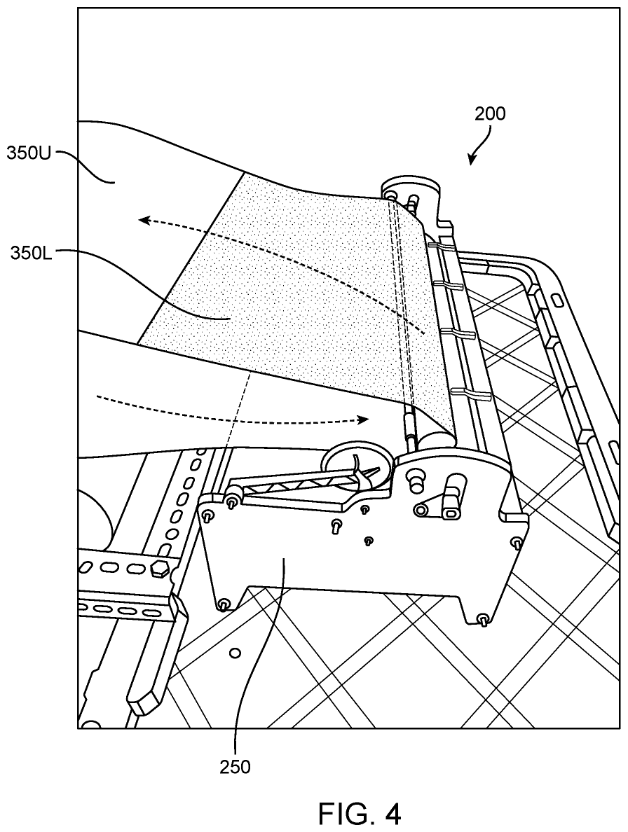

The paper 350 can then be brought into contact with the gel. As shown in FIG. 4, as the paper 350 is drawn through the gel which is in the basin 250 (as indicated by the dashed arrows), a layer of gel 350L is coated onto the paper. In this embodiment, the operator has advanced or positioned the paper through one or more rollers of the applicator assembly 200, so as to leave a portion 350U of the paper which has no gel. This can be done before lowering a top roller onto the paper. In other embodiments, hair styling aid can be applied to the paper via spraying or brushing.

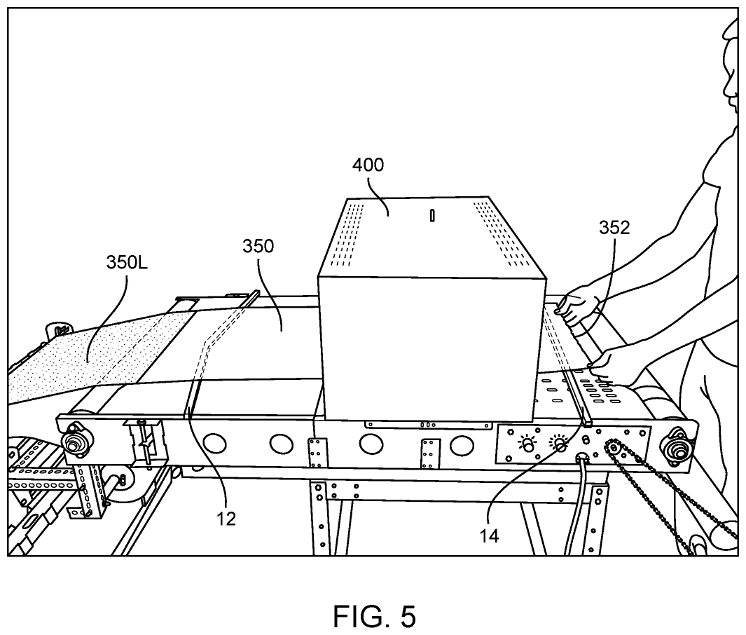

As shown in FIG. 5, the leading edge or portion of the paper 352 can be routed through the heating assembly 400. The system 10 may include supports 12, 14 which operate to keep the paper taut as it moves through the heating assembly 400. As the aid coated paper is advanced through or exposed to the heating assembly, the heating assembly can operate to remove water content from the aid which is present on the paper. According to some embodiments, the heating assembly 400 operates to expose the aid coated paper to a temperature of about 250 degrees Fahrenheit. During a manufacturing process, a thin layer of a non-stick material, such as a wax, can be present on the paper or embedded in the paper, and the presence of the hair styling aid which covers the non-stick material can operate to prevent the non-stick material from melting when the paper is exposed to the heat.

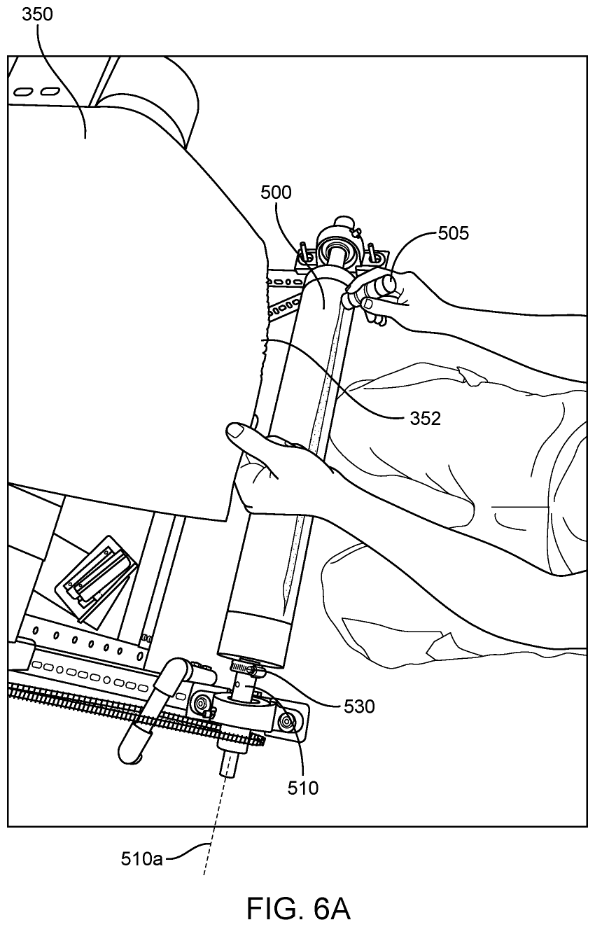

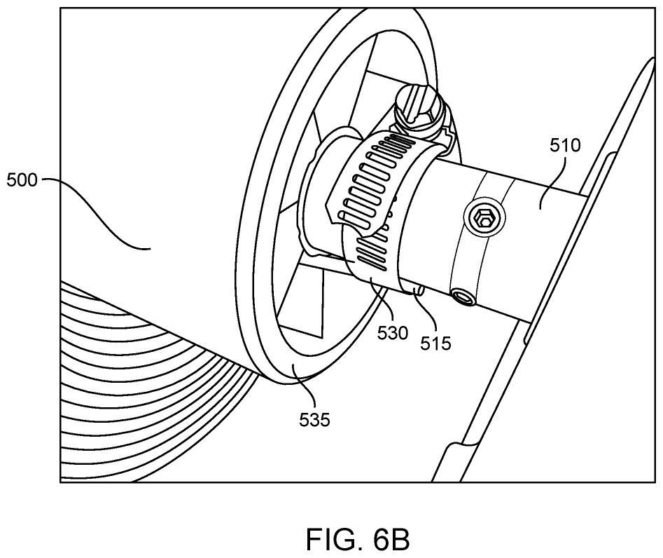

As shown in FIG. 6A, the leading edge of the paper 352 can be engaged with a receiving roller 500. In some cases, an adhesive such as a glue 505 (e.g. a temporary glue) can be applied to the receiving roller 500, so as to help maintain engagement between the paper leading edge 352 and the roller 500. In some cases, the roller 500 is engaged with or powered by a motor, which operates to turn the roller. As shown here, the roller 500 can include one or more lock mechanisms 530, for example engaged with a dowel 510 at one or both ends of the roller 500, so restrain the roller 500 from moving or translating along the long axis of the dowel 510. This can help to ensure alignment of the roller with other components of the system. According to some embodiments, while the heater assembly is warming up, the operator can finish pulling the paper slowly through the heater to the roller 500, which may be connected to a motor. The leading edge 352 can be attached with the roller 500 with a temporary adhesive so it is easy to remove later.

As illustrated here, the dowel 510 of the receiving roller 500 has a longitudinal axis 510a. In some embodiments, the paper holder assembly and the dowel 510 of the receiving roller 500 can be relative to one another so that a longitudinal axis of the dowel of the paper holder assembly and the longitudinal axis 510 of the dowel of the receiving roller 500 are in parallel relationship with each other. In some embodiments, the hair styling aid applicator assembly and the receiving roller can be aligned relative to one another so that a longitudinal axis of a roller of the hair styling aid applicator assembly and the longitudinal axis 510a of the dowel 510 of the receiving roller 500 are in parallel relationship with each other. In some embodiments, the paper holder assembly, the hair styling aid applicator assembly, and the receiving roller can be aligned relative to one another so that a longitudinal axis of the dowel of the paper holder assembly, a longitudinal axis of the roller of the hair styling aid applicator assembly, and the longitudinal axis 510a of the dowel 510 of the receiving roller 500 are in parallel relationship with each other.

The paper with the layer of gel can be placed in the heating assembly, and the heating assembly can be activated. Once the heating assembly warms up, the operator can turn on the motor to pull the gel coated paper through the heating assembly. In some cases, the operator may adjust the speed of the motor, which controls the rotational speed of the receiving roller. In some cases, the operator may wish to ensure that the uncoated portion of the paper is advanced through the heating assembly before the heating assembly becomes heated.

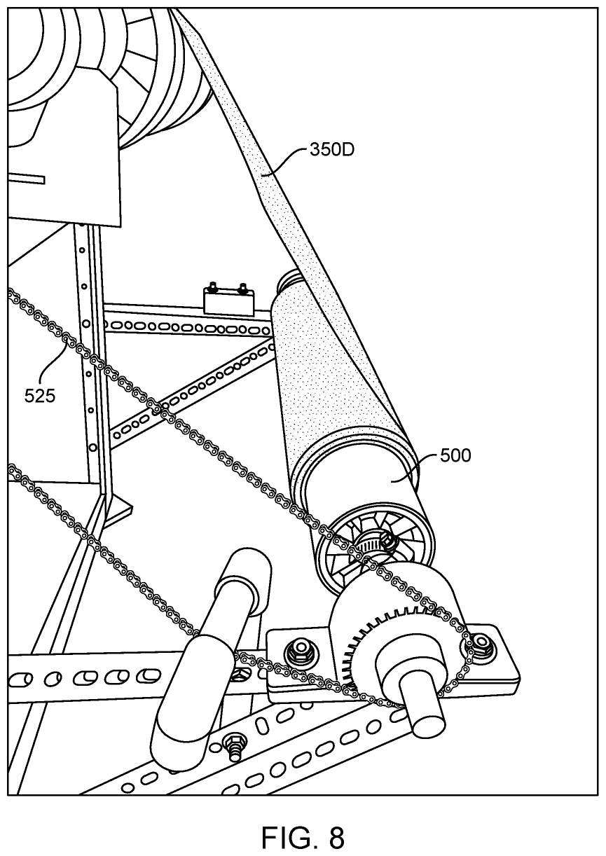

As shown in FIG. 6B, in some cases, the roller 500 can include one or more lock mechanisms 530, for example engaged with a dowel 510 at one or both ends of the roller 500, so restrain the roller 500 from moving or translating along the long axis of the dowel 510. Further, the roller 500 can include or be engaged with a drive assistance mechanism 515. The drive assistance mechanism 515 can be on the side of the dowel 510 which is located closest to the drive chain (e.g. drive chain 525 shown in FIG. 8). In the embodiment illustrated in FIG. 6B, the drive assistance mechanism 515 is provided as a small cylindrical rod, which engages the lock mechanism 530 (which may be a pipe clamp) as well as the end cap 535 of the roller 500, so as to ensure that when the dowel 510 turns (as driven by the drive chain), the roller 500 will also turn.

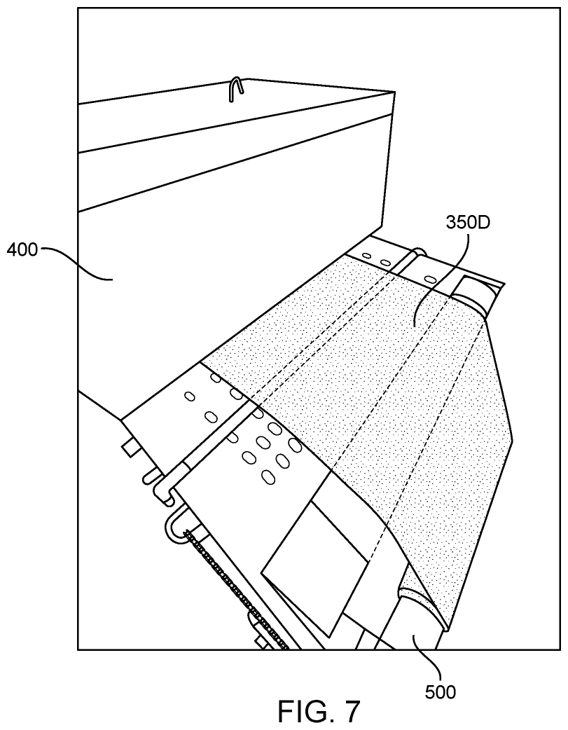

As shown in FIG. 7, as the receiving roller 500 continues to rotate, it continues to take up additional aid coated paper (i.e. which includes paper and a layer of aid) after the aid coated paper has passed through the heating assembly 400. In this way, the receiving roller 500 can be rotated so as to expose the aid coated paper to the heating assembly, thereby producing a dehydrated aid coated paper 350D. The dehydrated aid coated paper 350D can be dry and smooth to the touch, and not sticky and wet, or crusty and rough. FIG. 8 illustrates how the dehydrated aid coated paper 350D continues to wrap around the receiving roller 500. Operation of the drive roller 500 can be controlled by a motor (not shown) via a drive chain 525. In some cases, the aid coated paper may be advanced through or exposed to a dehydration mechanism (e.g. which operates to remove water content from the aid which is present on the paper). The speed of the drive chain 525 (or rotational speed of the roller 500) can be controlled so that as the roller continues to take up additional dehydrated aid coated paper 350D, the rotational speed becomes slower.

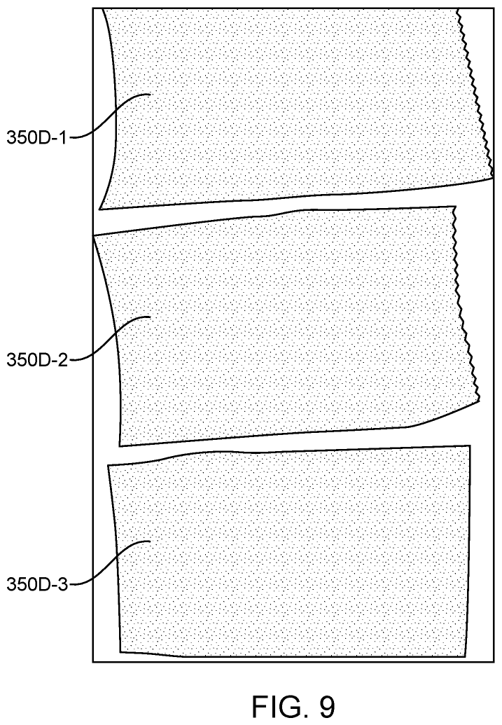

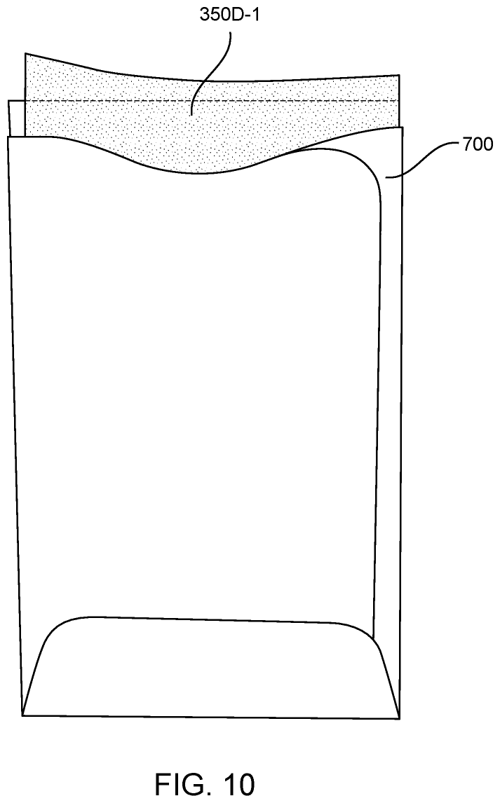

After the aid coated paper 350C passes through the heating assembly and has been wrapped about the receiving roller 500, the resulting roll of dehydrated aid coated paper 350D can be stored as desired, until additional processing is performed on the roll. For example, the roll can be unrolled and the dehydrated aid coated paper 350D can be cut into multiple pieces (e.g. 350D-1, 350D-2, 350D-3) or hair styling sheets, as shown in FIG. 9. An industrial paper cutter can be used for this cutting process. One or more individual hair styling sheets (e.g. 350D-1) can be placed into a package 700 for shipment or sale, as illustrated in FIG. 10.

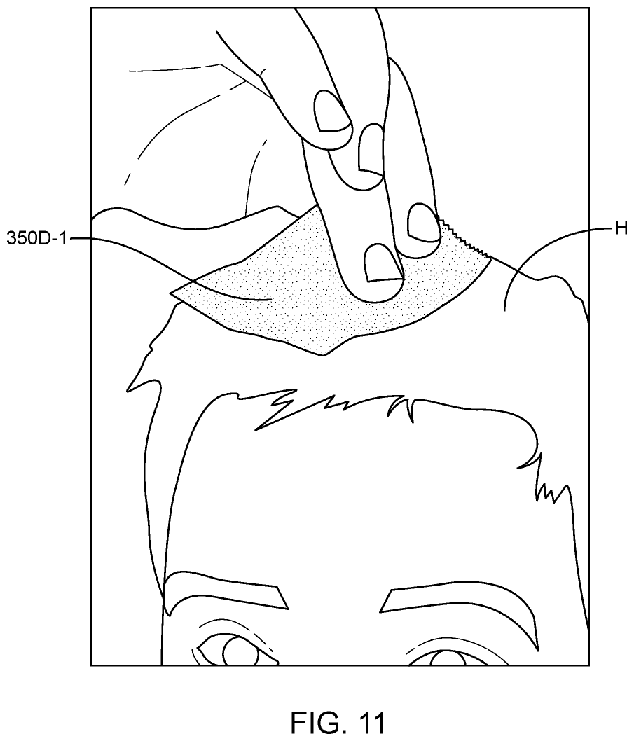

To use the hair styling sheet 350D-1, the user can apply the sheet 350D-1 to their hair, as shown in FIG. 11. In this way, the user can transfer the aid to their hair, and then dispose of the paper portion of the hair styling sheet. Often, the user will contact the sheet 350D-1 to their hair after they are finished taking a bath or a shower, so that their hair is wet when the sheet 350D-1 contacts their hair. Likewise, the user may opt to apply an amount of water to the sheet 350D-1 and then contact the moistened sheet to their hair, even when their hair is in a dry state. In some embodiments, a non-stick material which is present on or in the paper can operate to facilitate the application of the aid, because the aid may slide off the paper more easily when the non-stick material is present.

Use of a hair styling sheet as disclosed herein can eliminate the need to for the user to purchase a bottle of or tube of aid. Similarly, use of the hair styling sheet allows the user to apply aid to their hair without getting aid on their hands.

Although the preceding description contains significant detail in relation to certain preferred embodiments, it should not be construed as limiting the scope of the invention but rather as providing illustrations of the preferred embodiments.

Embodiments of the present invention encompass kits having one or more components of a system as disclosed herein. In some embodiments, the kit includes one or more system components, along with instructions for using the component(s) for example according to any of the methods disclosed herein.

All features of the described systems and devices are applicable to the described methods mutatis mutandis, and vice versa.

In addition, each reference provided herein in incorporated by reference in its entirety to the same extent as if each reference were individually incorporated by reference. Relatedly, all publications, patents, patent applications, journal articles, books, technical references, and the like mentioned in this specification are herein incorporated by reference to the same extent as if each individual publication, patent, patent application, journal article, book, technical reference, or the like was specifically and individually indicated to be incorporated by reference.

While preferred embodiments of the present disclosure have been shown and described herein, it will be understood to those skilled in the art that such embodiments are provided by way of example only. Numerous variations, changes, and substitutions will now occur to those skilled in the art without departing from embodiments of the present invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A hair styling sheet, comprising:
paper; and
hair styling aid.

2. The hair styling sheet according to claim 1, wherein the sheet has a dimension of 4 inches by 6 inches.

3. The hair styling sheet according to claim 1, wherein the hair styling aid comprises a member selected from the group consisting of a hair styling mousse, a gel, a hairspray, and a wax product.

4. The hair styling sheet according to claim 1, wherein the hair styling aid comprises a gel, and wherein the gel comprises a polymer.

5. The hair styling sheet according to claim 1, wherein the hair styling aid comprises a gel, and wherein the gel comprises a cationic polymer.

6. The hair styling sheet according to claim 1, wherein the hair styling aid comprises a gel, and wherein the gel comprises a copolymer.

7. A method of manufacturing a hair styling sheet, the method comprising:
placing a roll of paper on a dowel of a paper holder assembly;
placing a fluid hair styling aid into a basin of a hair styling aid applicator assembly;
contacting paper of the roll of paper with a roller of the hair styling aid applicator assembly;
positioning paper of the roll of paper adjacent to a heating assembly;
attaching a leading portion of the paper with a receiving roller;
contacting paper of the roll of paper with the fluid hair styling aid which is in the basin of the hair styling aid applicator assembly, so as to provide a layer of the fluid hair styling aid onto the paper, thus producing a fluid aid coated paper;
rotating the receiving roller so as to expose the fluid aid coated paper to the heating assembly and thereby produce a dehydrated aid coated paper, and so as to wrap the dehydrated aid coated paper around the receiving roller;
removing the wrapped dehydrated aid coated paper from the receiving roller; and
cutting a piece from the dehydrated aid coated paper, wherein the piece is the hair styling sheet.

8. The method according to claim 7, wherein the hair styling sheet has a dimension of 4 inches by 6 inches.

9. The method according to claim 7, wherein the fluid hair styling aid comprises a hair styling gel.

10. The method according to claim 7, wherein the fluid hair styling aid comprises a hair styling gel, and wherein the hair styling gel comprises a polymer.

11. The method according to claim 7, wherein the fluid hair styling aid comprises a hair styling gel, and wherein the hair styling gel comprises a cationic polymer.

12. The method according to claim 7, wherein the fluid hair styling aid comprises a hair styling gel, and wherein the hair styling gel comprises a copolymer.

13. The method according to claim 7, wherein the fluid hair styling aid comprises a hair styling gel, and wherein the hair styling gel comprises a viscosity modifier.

14. The method according to claim 13, wherein the viscosity modifier comprises a carbomer.

15. The method according to claim 7, wherein the fluid hair styling aid comprises a hair styling gel, and wherein the hair styling gel comprises a fragrance.

16. The method according to claim 7, wherein the fluid hair styling aid comprises a hair styling gel, and wherein the hair styling gel comprises an ultraviolet protectant.

17. The method according to claim 7, further comprising aligning the paper holder assembly and the hair styling aid applicator assembly relative to one another so that a longitudinal axis of the dowel of the paper holder assembly and a longitudinal axis of the roller of the hair styling aid applicator assembly are in parallel relationship with each other.

18. The method according to claim 7, further comprising aligning the paper holder assembly and a dowel of the receiving roller relative to one another so that a longitudinal axis of the dowel of the paper holder assembly and a longitudinal axis of the dowel of the receiving roller are in parallel relationship with each other.

19. The method according to claim 7, further comprising aligning the hair styling aid applicator assembly and the receiving roller relative to one another so that a longitudinal axis of the roller of the hair styling aid applicator assembly and a longitudinal axis of a dowel of the receiving roller are in parallel relationship with each other.

20. The method according to claim 7, further comprising aligning the paper holder assembly, the hair styling aid applicator assembly, and the receiving roller relative to one another so that a longitudinal axis of the dowel of the paper holder assembly, a longitudinal axis of the roller of the hair styling aid applicator assembly, and a longitudinal axis of a dowel of the receiving roller are in parallel relationship with each other.
